(19) United States
(12) Patent Application Publication
(10) Pub. No.: US 2010/0241002 A1
(43) Pub. Date: Sep. 23, 2010

(54) 2D Ultrasound Transducer for Radial Application and Method

(75) Inventor: Wojtek Sudol, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/090,389

(22) PCT Filed: Oct. 18, 2006
(86) PCT No.: PCT/IB2006/053842

§ 371 Date: Apr. 16, 2008

Related U.S. Application Data
(60) Provisional application No. 60/728,387, filed on Oct. 19, 2005.

(51) Int. Cl. A61B 8/14

(52) U.S. Cl. 600/459

(57) ABSTRACT

The present invention relates to an apparatus and a method for obtaining a 3D image in radial applications, typically endorectal imaging.

The present invention relates to an apparatus and a method for obtaining a 3D image in radial applications, typically endorectal imaging where the object of interrogation is the rectal wall. In particular, the present invention is related a two dimensional (2D) acoustic array transducer that is wrapped around a cylindrically shaped probe so that the 2D array is capable of steering the beam radially and axially to obtain a precise 3D data acquisition of the object of interrogation.

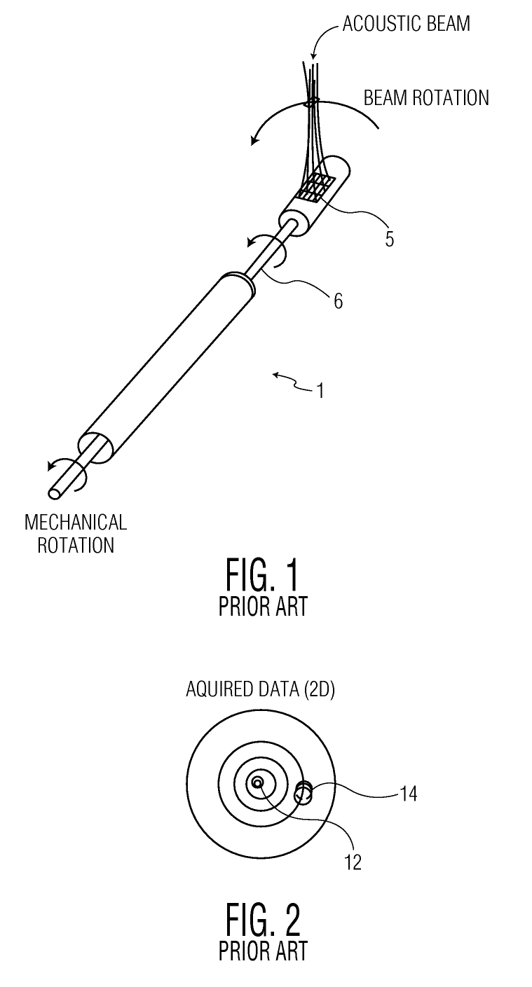

The prior art transducer (see FIG. 1) requires coordination of rotational and translational movement of the transducer by the operator e.g. physician or technician. These movements are very difficult to achieve and can vary in result based on the coordination and skill of the operator as well as due to human error in moving the probe.

WO2005/053863A1 (by the same inventor and same assignee as for the present application) discloses bending a flip chip two dimensional array. This reference does not disclose bending the flip chip 360 degrees or use in endorectal imaging.

The present invention provides for obtaining a 3D image in one acquisition by bending an acoustic array of a flip chip, acoustic transducer 360 degrees and mounting it on a cylindrical probe. The present invention provides for 3D image acquisition for endorectal imaging using a 2D transducer that eliminates the need for the operator to provide rotational and translational movement of the cylindrical probe in order to obtain a 3D acquisition of the rectal wall.

FIG. 1 is a perspective view of a prior art acoustic transducer probe;

FIG. 2 is an image of an area of interrogation rotating the probe of FIG. 1 360 degrees to acquire data;

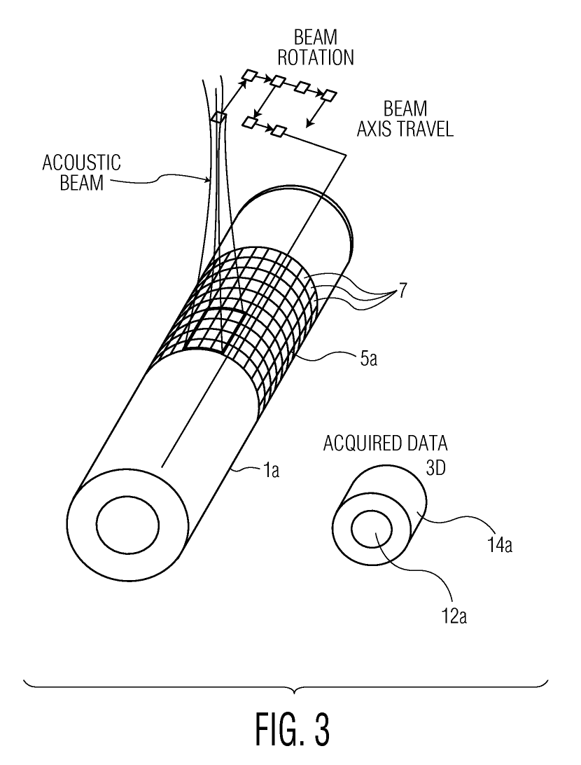

FIG. 3A is a perspective view of the present invention;

FIG. 3B is an image of an area of interrogation in 3D using the probe of the present invention of FIG. 3A;

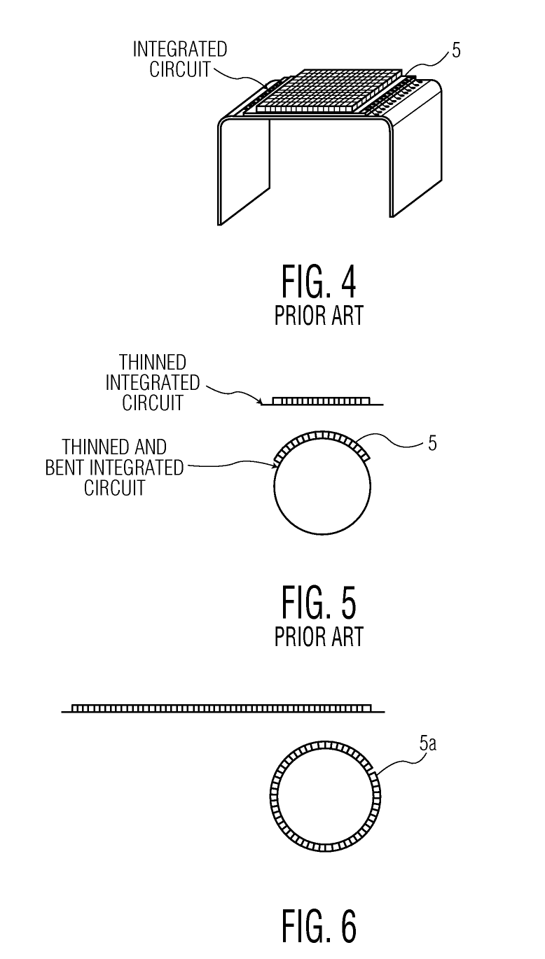

FIG. 4 shows a known flip-chip transducer;

FIG. 5 illustrates a thinned and bent flip-chip transducer; and

FIG. 6 is the radial transducer of the present invention to be mounted on the cylindrical probe so as to appear as shown in FIG. 3A.

Referring to the drawings of FIGS. 1-6, FIG. 1 illustrates a prior art transducer probe 1. The transducer 5 is a two dimensional (2D) acoustic array. It is necessary for the operator e.g. physician or technician to provide rotational and translational motion for obtaining a three dimensional image in radial applications such as for endorectal imaging.

The cylindrical shaft is shown in a partially exploded perspective view to show the shaft 6 on which the cylinder is mounted on.

FIG. 2 shows the acquired two dimensional data from the prior art transducer of FIG. 1 where element 12 shows the center and element 14 shows detected structure.

FIG. 3A shows the present art in which a two dimensional acoustic transducer 5a is formed in a 360 degree bent shape to encircle the cylindrical probe 1a on which it is mounted.

FIG. 3B illustrates the acquired three dimensional data from the present invention shown in FIG. 3A in which X, Y, and Z coordinates identify the location of the center 12a and an element 14a showing detected structure.

FIG. 4 is a typical flip-chip transducer 5 known in the art.

FIG. 5 illustrates the transducer 5 of FIG. 4 being thinned and bent as described in the aforementioned reference WO2005/05 3863A1.

FIG. 6 illustrates the 360 degree shaped flip-chip transducer 5a of the present invention—a radial transducer.

The cylindrical two dimensional array 5 can be manufactured using flip-chip technology where the beam forming circuitry resides in the IC and the acoustic elements 7 (see FIG. 3) are positioned and electrically attached directly to the IC circuitry.

The ASCIC silicon material of the flip-chip transducer 5 has become flexible due to the thinning process and can be reshaped into a circular or a substantially circular shape. This is done by a thinning process of polishing chemical etching, plasma etching, or a combination thereof. After dicing operation (that separate the slab of material into individual elements) the assembly (IC and acoustic elements) will be very flexible and can be bent to the desired curvature appropriate for different applications.

The thickness of the IC has to be reduced to a range of 7-50 microns. At this range of thickness the IC is becoming flexible. The thinning range for the radial transducer 5a of the present invention is 20 microns to 80 microns.

The circularly shaped transducer 5a of the present invention is mounted and affixed onto a cylindrical probe 1a (as shown in FIG. 6) by adhesive means such as epoxy. The result is a transducer for obtaining 3D images in one acquisition in radial applications such as endorectal imaging.

In addition to being used as an endorectal radial transducer, the present invention can also be used as an intracardiac transducer (ICE).

By wrapping the two dimensional transducer array 5a around an axis of symmetry e.g. the cylindrical probe 1a, a cylindrical array is created by the present invention.

The 2D array is thus capable of steering the beam radially and axially to enable precise 3D data acquisition. In this way, the present invention provides one with the ability to use a large number of elements 7 in the 2D array enabling superior beam focusing and improved near field image equality.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. An apparatus for acquiring a three dimensional image in radial applications; comprising:
a two dimensional acoustic array transducer bent and shaped 360 degrees into a circular shape; and
a cylindrical probe adapted to have said 360 degree shaped two dimensional acoustic array mounted and affixed thereon so as to provide for precise three dimensional (3D) data acquisition of an area of interrogation.

2. The apparatus according to claim 1 wherein said transducer is made of flip-chip technology and said transducer is thinned to bend and shape said transducer into a 360 degree circular shape.

3. The apparatus according to claim 2 wherein said transducer is thinned within a range of 20 microns to 80 microns.

4. The apparatus according to claim 2 wherein said transducer is mounted and affixed to said cylindrical probe by epoxy.

5. The apparatus according to claim 1 wherein said apparatus is for endorectal imaging area of interrogation is rectal walls of a patient.

6. The apparatus according to claim 1 wherein said transducer is for use as an intracardial transducer

7. A method for acquiring a three dimensional image in radial applications, the steps comprising:
bending and shaping a two dimensional acoustic array transducer 360 degrees into a substantially circular shape; and
mounting and affixing said 360 degree bent and shaped transducer onto a cylindrical probe so as to provide for precise three dimensional (3D) data acquisition of an area of interrogation.

8. The method according to claim 7 wherein said radial application is for endorectal imaging and said area of interrogation for rectal walls of a patient.
